(19) United States
(12) Patent Application Publication
(10) Pub. No.: US 2019/0029760 A1
(43) Pub. Date: Jan. 31, 2019

(54) Totally Implantable Vascular Access Device With Integrated Subcutaneous Localization System

(71) Applicant: PORTSIGHT MED LTD., Tel-Aviv (IL)

(72) Inventors: Dean Nahman, Jerusalem (IL); Allan Bloom, Neve Ilan (IL); Hamutal Eytan, Tel-Aviv (IL)

(21) Appl. No.: 16/072,914

(22) PCT Filed: Jan. 31, 2017
(86) PCT No.: PCT/IB2017/050510

§ 371 Date: Jul. 26, 2018

Related U.S. Application Data
(60) Provisional application No. 62/289,354, filed on Feb. 1, 2016.

(51) Int. Cl. A61B 34/20; A61F 2/06; A61M 1/36; A61M 39/02; A61B 90/00; A61B 17/34; A61B 1/07

(52) U.S. Cl. CPC A61B 34/20; A61F 2/06; A61M 1/3661; A61M 39/0208; A61B 90/02; A61M 2039/0238; A61B 1/07; A61B 2090/3937; A61B 2034/2055; A61F 2250/0097; A61B 17/3403

(57) ABSTRACT

A totally implantable medical device configured to be implanted under a patient's dermal layer for vascular access comprising: a housing comprising a reservoir; said reservoir connected on one end thereof to a self-resealing penetrable septum and on the other end to an outlet cannula; and one or more inter-digitating light carrying channels connected with said housing having their openings circumferentially scattered about the outer rim of said septum, the device configured to be located using a generic light source.

### CROSS-REFERENCE TO RELATED PATENT APPLICATIONS

This patent application claims priority from and is related to U.S. Provisional Patent Application Ser. No. 62/289,354, filed 1 Feb. 2016, this U.S. Provisional patent application incorporated by reference in its entirety herein.

### FIELD OF THE INVENTION

The present invention relates to the field of medical devices for vascular access. More particularly, the present invention relates to a novel totally implantable vascular access device (TIVAD) with integrated sub dermal localization apparatus.

### BACKGROUND OF THE INVENTION

Central venous access devices (CVAD) are widely used all over the world. These devices are especially used among oncology patients who undergo cyclical long term chemotherapeutic treatments, extended antibiotic therapy and total parenteral nutrition.

TIVADs such as Port-A-Cath (PAC) are a type of CVAD totally implanted under the patient skin usually on the pectoral region. A PAC insertion in the venous system is a surgical procedure, requiring local anesthesia, performed by interventional radiologists and surgeons. After being surgically inserted the reservoir (or lumen) within the device must be filled with therapeutic agents for carrying out the medical treatments. The device is accessed percutaneously using a specialized non-coring Huber needle with standard sterile technique.

A peripherally inserted central catheter (PICC or PIC line) is a CVAD inserted through a peripheral vein, usually in the ante-cubital fossa. The distal end of the PICC is located in the right atrium of the heart while the proximal end, to which the infusion equipment connects is left exposed.

PICC lines and TIVADs have many advantages over other central venous catheters and intravenous lines; however their usage is impaired due to several complications and drawbacks. PICC lines exposed end causes several major drawbacks—accidental withdrawal rate of 6%, an infection rate of 8%, and they require frequent expensive dressing replacement.

A major shortcoming of accessing a TIVAD is due to the fact that the PAC is located by palpation, so the TIVAD must have a minimum size to be located. Mainly due to their size, TIVADs are implanted in an invasive time consuming and painful procedure, with a perioperative infection rate of 4%, 5-10% of patients suffer additional late infections. About 1% of implanted TIVADs are not suitably stabilized to the surrounding tissue and invert, leading to unsuccessful needle insertions, a need for X-ray imaging and costly re-implantations. 40-70% of the patients are dissatisfied with cosmetic outcome, suffer pain, discomfort and fear needle insertions.

It is therefore an object of the present invention to provide methods and means for a novel TIVAD system with integrated subdermal localization system aiming to reduce complications and drawbacks of conventional TIVADs and PICC lines.

Other objects and advantages of the present invention will become apparent as the description proceeds.

### SUMMARY

According to a first aspect of the present invention there is provided a totally implantable medical device configured to be implanted under a patient's dermal layer for vascular access comprising: a housing comprising a reservoir; said reservoir connected on one end thereof to a self-resealing penetrable septum and on the other end to an outlet cannula; and one or more inter-digitating light carrying channels connected with said housing having their openings circumferentially scattered about the outer rim of said septum.

The one or more inter-digitating light carrying channels may be configured to internally transmit light rays by means of internal reflection.

The one or more inter-digitating light carrying channels may comprise optic fibers.

The one or more inter-digitating light carrying channels may comprise light guides.

The septum may be located on the side or upper surface of said housing.

The medical device may further comprise integrated stabilization wings.

The medical device may be configured to be located using a generic light source.

According to another aspect of the present invention there is provided an implantation device configured to implant a sub dermal medical device comprising: a body; a needle connected to said body; means for keeping a precise implantation depth and an operation button located on said body.

According to another aspect of the present invention there is provided a designated access device configured to locate an implanted medical device according to claim 1, comprising: a body; a needle connected to said body; and integral light source connected to said body.

According to another aspect of the present invention there is provided a designated light emitting device configured to mark the septum of an implanted medical device according to claim 1, comprising: a sterile patch; said patch comprises outer orifices and an inner orifice; and a light source located on said patch.

According to another aspect of the present invention there is provided a sub-dermal bundle of optical fibers configured to mark a desired location on the skin under induction of an external light source.

According to another aspect of the present invention there is provided a method of locating a medical device comprising inter-digitating light carrying channels under a patient's dermal layer for vascular access, comprising: inducing light with a light source on said patient's skin; and finding a reflected light area on said patient's skin.

According to another aspect of the present invention there is provided a method of implanting a sub dermal medical device, comprising: inserting manually a catheter to a medical device; loading said connected catheter and medical device to an implantation device; puncturing the skin with a needle of said implantation device; inserting said medical device; and withdrawing said needle.

According to another aspect of the present invention there is provided a method of locating a medical device comprising inter-digitating light carrying channels under a patient's dermal layer, comprising: holding a light emitting sterile patch near a patient's skin; searching for reflected light; and applying said patch to said patient's skin when light is noticed.

According to another aspect of the present invention there is provided a method of locating a desired location on the skin, comprising: inserting sub-dermally a bundle of optical fibers; inducing an external light source; and searching for reflected light.

According to another aspect of the present invention there is provided a method of locating a totally implantable vascular access device (TIVAD) comprising inter-digitating light carrying channels under a patient's dermal layer, comprising: launching a dedicated application running on a smartphone and operable to: collect information when the TIVAD is in use; illuminate the skin using the smartphone's LED; detect the TIVAD; capture the illuminated septum area; analyze the captured image; and navigate an injecting device to areas less dense with previous injections.

The information may comprise at least one of type of drug injected, date and time.

The illuminating may comprise attaching a light guide to the LED phone flash.

### BRIEF DESCRIPTION OF THE DRAWINGS

For better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

The present invention is illustrated by way of example in the accompanying drawings, in which similar references consistently indicate similar elements and in which:

FIG. 1 schematically illustrates an exterior view of an embodiment of the present invention;

FIG. 2 schematically illustrates a mid-sectional view of an embodiment of the present invention;

FIG. 3 shows an example of an embodiment of the present invention;

FIG. 4 shows an example wherein the embodiment of the present invention shown in FIG. 3 is used for determining location and orientation of the PAC device of the invention;

FIG. 5 schematically illustrates an outside view of an embodiment of the present invention;

FIG. 6 schematically illustrates a mid-sectional view of an embodiment of the present invention;

FIG. 7A shows the TIVAD device of the present invention installed as an add-on to an existing conventional TIVAD;

FIG. 7B shows the target shape illuminated in various shapes;

FIG. 8 shows an integrated injection device and light emitting needle;

FIGS. 9A-9B schematically illustrates a designated light emitting device;

FIG. 9C shows a light emitting device is attached to the physician's hand;

FIG. 10 shows use of a smartphone to provide the required illumination;

FIGS. 11A-11E schematically illustrate a designated implantation device for precise subcutaneous implantation of the port; and

FIGS. 12A-12J show various exemplary designs of the TIVAD according to the present invention.

It is noted that the embodiments exemplified in the Figures. Are not intended to be in scale and are in diagrammatic form to facilitate ease of understanding and description.

### DETAILED DESCRIPTION OF THE INVENTION

The present invention relates to a novel TIVAD which can be illuminated trans-cutaneously prior to needle insertion thereby indicating to the medical personnel the presence or absence of appropriate TIVAD orientation and location for safe access, thus allowing the medical personnel to determine whether to alter the position, or move the location, of the TIVAD as a result of viewing the TIVAD orientation and location.

It is noted that the prescribed subcutaneous localization device of the present invention can be used for other applications and devices such as—tissue expander, other vascular access devices, vascular grafts, dialysis/surgical bypass grafts or any other sub cutaneous devices.

In an embodiment of the present invention the TIVAD comprises a standard TIVAD device and further comprises trans-cutaneous light emitting means that allow the membrane to be reliably and repeatedly located by the trans-cutaneous illumination of the TIVAD, thus enabling an easy and safe access to the TIVAD for therapeutic agent delivery.

In an embodiment of the present invention, the TIVAD light emitting means are conductive optic fibers. The optic fibers are incorporated into the casing of the TIVAD in an orientation that allows the membrane (septum) to be reliably and repeatedly located by trans-cutaneous illumination of the TIVAD using a commercially available monochromatic laser light source, or any other light source.

According to embodiments of the invention, the TIVAD light emitting means may be selected from any optic conductors known in the art, such as full glow cables, end glow cables and engraved optic boards.

It is of importance to stress out that light reflectance and guiding may comprise other means than optic fibers, such as, acrylic glass (PMMA), mirrors or other light reflecting materials.

According to embodiments of the invention the TIVAD light emitting method may be selected from any means known in the art such as phosphorescense materials, photoluminescence materials reflecting UV light, IR reflecting materials and materials capable of absorbing IR wavelength and re-emitting visible light.

According to embodiments of the invention, detecting the TIVAD may also be done using thermal imaging.

According to embodiments of the invention, detecting the TIVAD may also be done using RFID means implanted in the device.

The TIVAD design of the present invention allows minimizing the TIVAD size without compromising the ease of septum localization. Furthermore, the subcutaneous localization system allows a novel ultra-low profile design for the TIVAD that can be implanted using a less invasive procedure, perhaps by a designated nurse at a bed side setting.

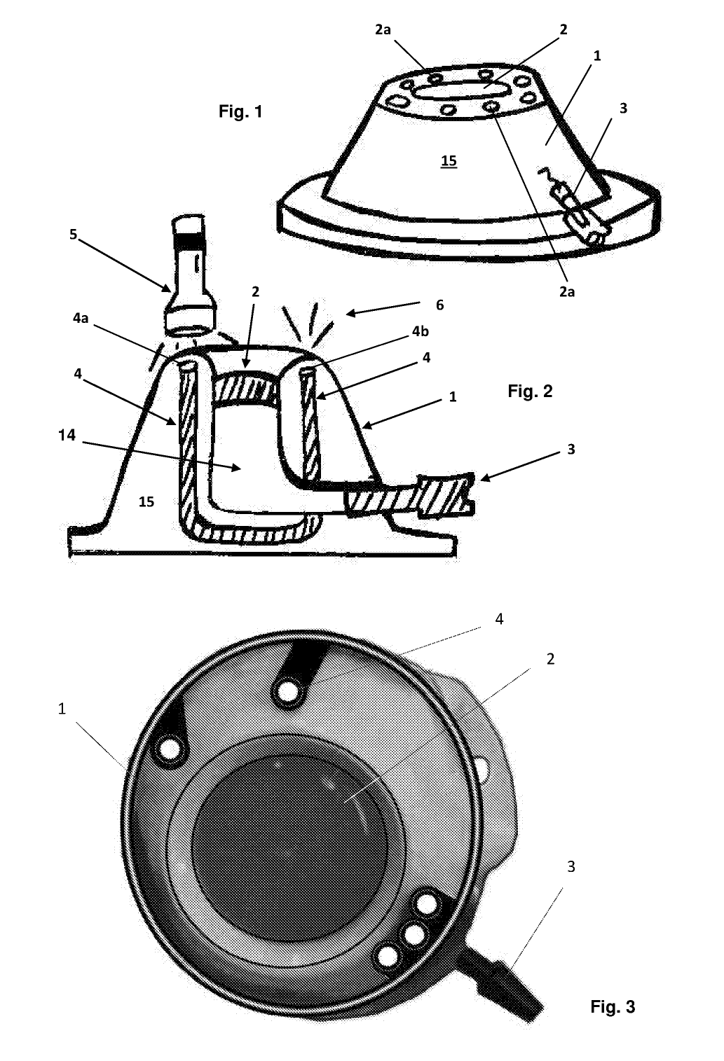

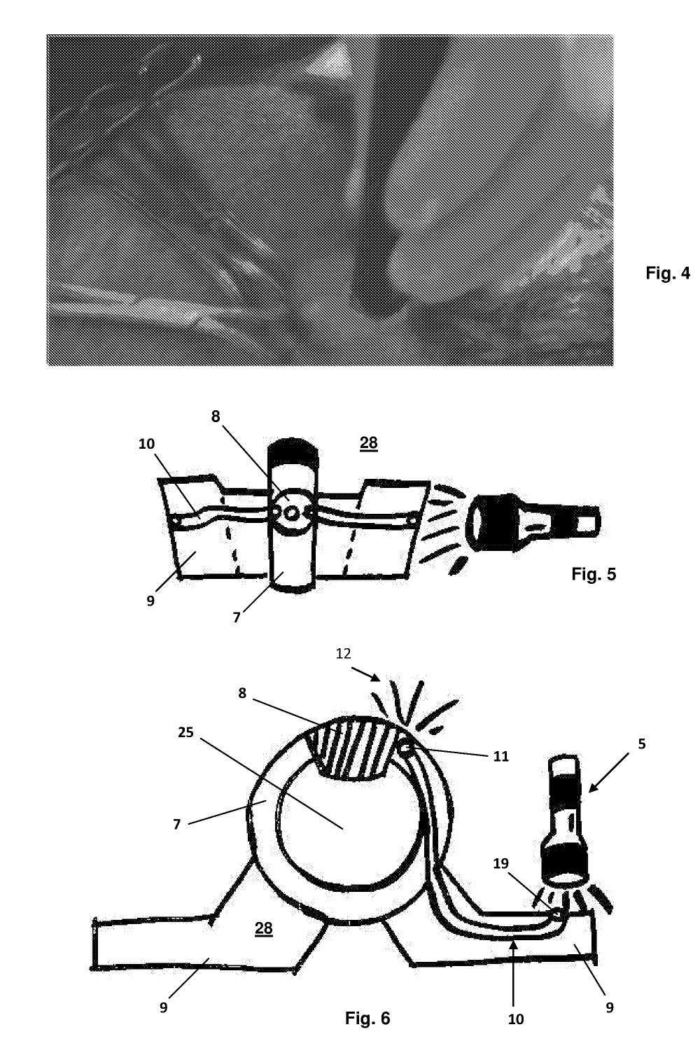

FIG. 1 schematically illustrates an exterior view of the implantable illuminating TIVAD 15 of the present invention. The basic structure of the TIVAD device 15 of the present invention is substantially similar to that of a conventional commercial TIVAD. The housing 1 of TIVAD device 15 includes an interior reservoir 14 (FIG. 2), which is spanned by a self resealing penetrable septum 2, which can be located on the side or upper surface of the TIVAD. At one point reservoir 14 is connected to outlet cannula 3, which is used for leading injected fluid towards the vascular system. TIVAD device 15 further comprises one or more inter-digitating light carrying channels 4 (FIG. 2) i.e., capable of internally transmitting light rays by means of internal reflection (e.g., optic fibers) having their openings 2a circumferentially scattered about the outer rim of septum 2.

FIG. 2 shows a mid-sectional view of the present invention TIVAD device. One end 4a of the optic fiber 4 is located at the TIVAD's rim, while the other end 4b is located at the opposite side, creating a U shaped figure, wherein both ends of the U shaped figure are placed on the outer rim of the septum 2 as shown in FIG. 1. When illuminating through the skin using a light source 5 at one end 4a of the fiber 4, the other end 4b will be simultaneously illuminated (indicated by reference 6), facilitating TIVAD detection and orientation.

In an embodiment of the invention each light carrying channel 4 is accessible via two opposing openings, 4a and 4b, provided on the upper circumference of housing 1, such that any light rays entering a light carrying channel 4 via one of its openings 4a will effectively pass through it and illuminate via the opening 4b at its other end. However, in alternative embodiments (not illustrated) of the invention the light carrying channels 4 may be optically coupled (the fibers interconnected), such that light rays entering one opening are scattered among several, or all, of the light carrying channels such light rays are illuminated via several openings provided on the upper circumference of housing 1.

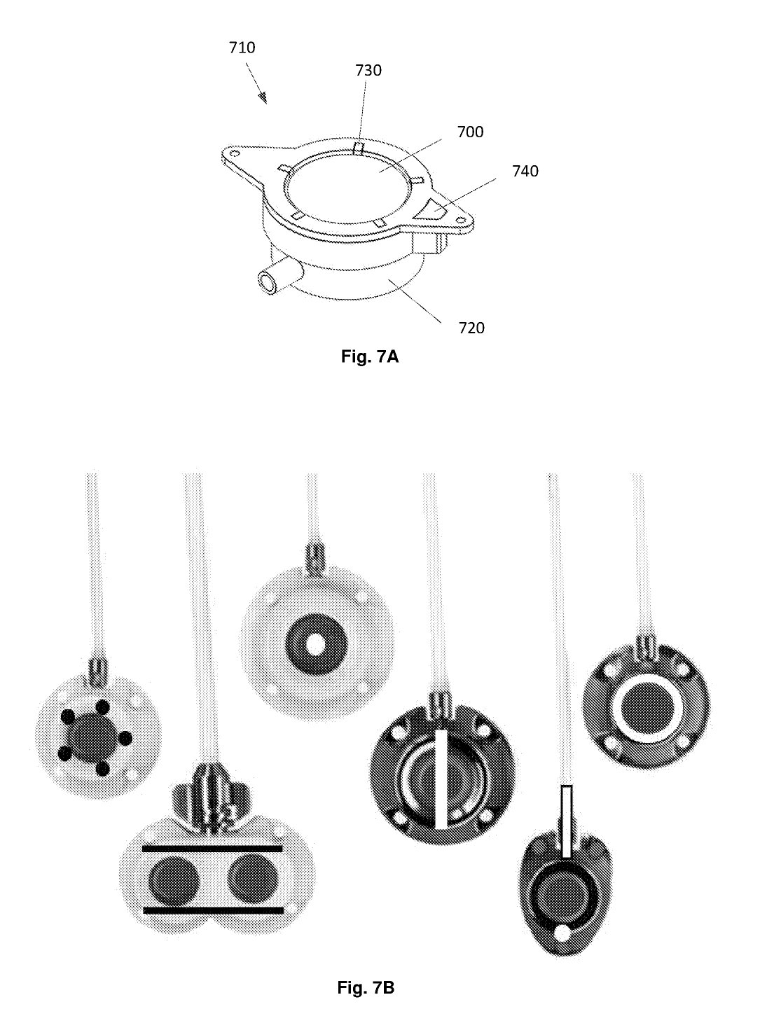

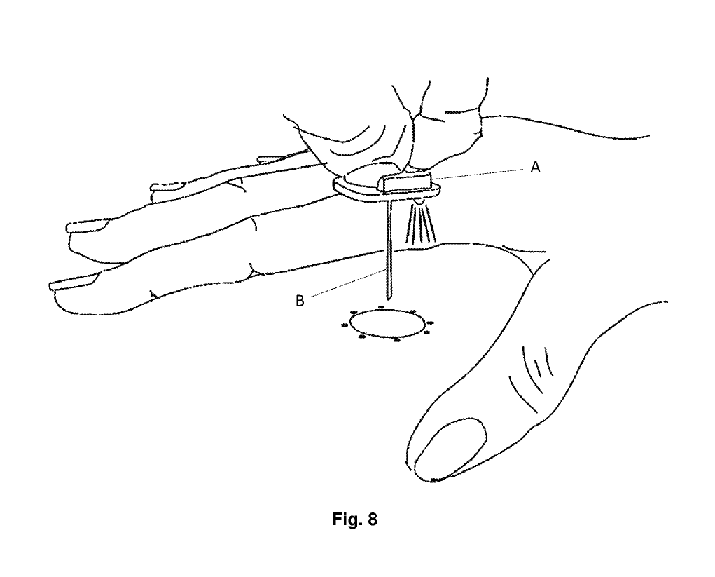

FIG. 3 shows an example of an embodiment of the present invention. In this embodiment the TIVAD device 1 (having diameter of about 2.5 cm, height of about 1 cm, and wall thickness of about 3 mm) was designed to comprise three thin optic fibers 4 having diameter of about 1 mm, and was implanted subcutaneously into a deceased miniature pig (16 Kg weight) at three different depths from the skin surface. At 2-3 mm below the surface the optic fibers 4 were clearly illuminated by the application of a standard laser pointer applied at the skin surface as shown in FIG. 4, thereby indicating the location and orientation of septum 2 (having diameter of about 2 cm). Placement 2-3 mm below the skin surface correlates to current clinical practice for port insertion, thus indicating that the simple prototype shown in FIG. 3 provides sufficient enablement for carrying out the invention.

TIVAD device 15 may be manufactured employing similar materials and techniques conventionally used in the PAC devices manufacturing industry. The wall thickness of housing 1 of TIVAD device 15 should be however configured to include light carrying channels 4 (e.g., fiber optics). For example, the wall thickness of housing 1 may generally be in the range of 2 to 4 mm, preferably about 3 mm. The diameter of the light carrying channels 4 may generally be in the range of 0.5 to 2 mm, preferably about 1 mm. It should however be clear that the geometrical dimension of the TIVAD device 15 may be varied in accordance with the specific implementation, and that other changes may be made without departing from the scope of the present invention. For example, the wall thickness of TIVAD device 15 may be the same as that of conventional PAC devices by implementing the light carrying channels 4 by means of optical fibers attached over the outer surface of the TIVAD device, as exemplified in FIG. 3.

In another embodiment of the present invention of the TIVAD device, there is the ability to detect the device using a trans-dermal light source by means of the optic fibers configuration, as described herein above, as opposed to locating by palpation, thus its size may be reduced in comparison to conventional PAC devices, and it allows a smaller surgical incision for placement or even a bedside implantation using a designated implantation device.

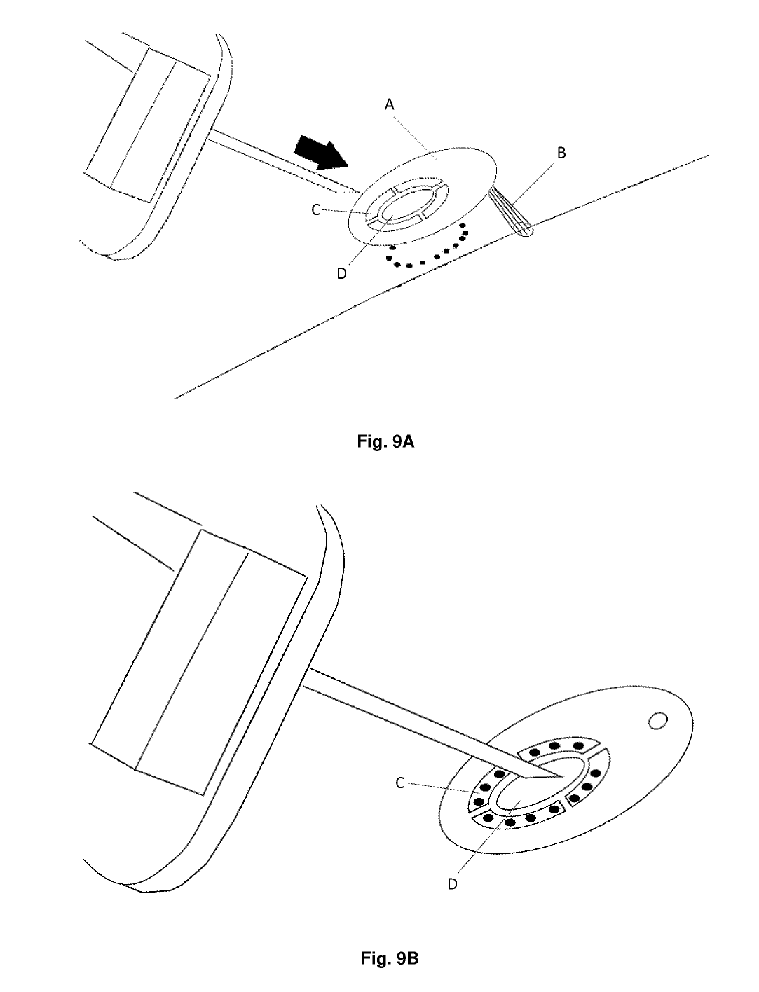

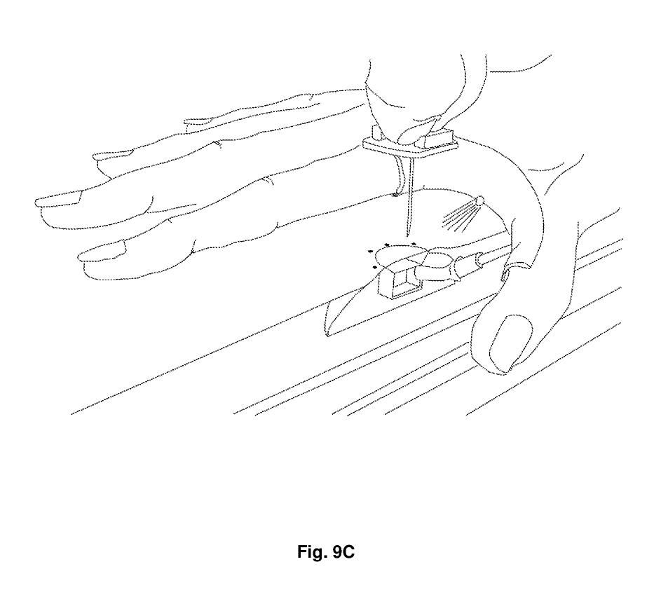

FIG. 5 schematically illustrates an outside view of such a device 28. TIVAD device 28 comprises a small cylindrical housing 7 comprising an interior reservoir 25 (shown in FIG. 6), which is spanned by a self resealing penetrable septum 8. The housing is held in place by two or more (two shown) foldable wings 9. Light carrying channels 10 (e.g., Optic fibers) are integrated in each foldable wing (or any other location) 9, such that one of their openings 11 (FIG. 6) is located adjacent to septum 8.

FIG. 6 shows a mid-sectional view of the TIVAD device 28 having reduced size according to the invention. As illustrated, one opening 19 of the light carrying channel 10 (optic fiber) is located at the distal (remote) end of foldable wing 9, while the other opening 11 is located adjacent to the rim of the septum 8. When introducing light by a light source 5 through the skin to the opening 19 provided at the distal end of foldable wing 9, the light rays are transferred via light carrying channel 10 to the other opening 11, which therefore glows (indicated by numeral reference 12), thereby making septum 8 detectable. This enables introduction of the light source 5 even at a further distance from membrane 8 than as explained herein above in other embodiments. According to embodiments of the invention, a plurality of light carrying channels, may be located in any part of the TIVAD.

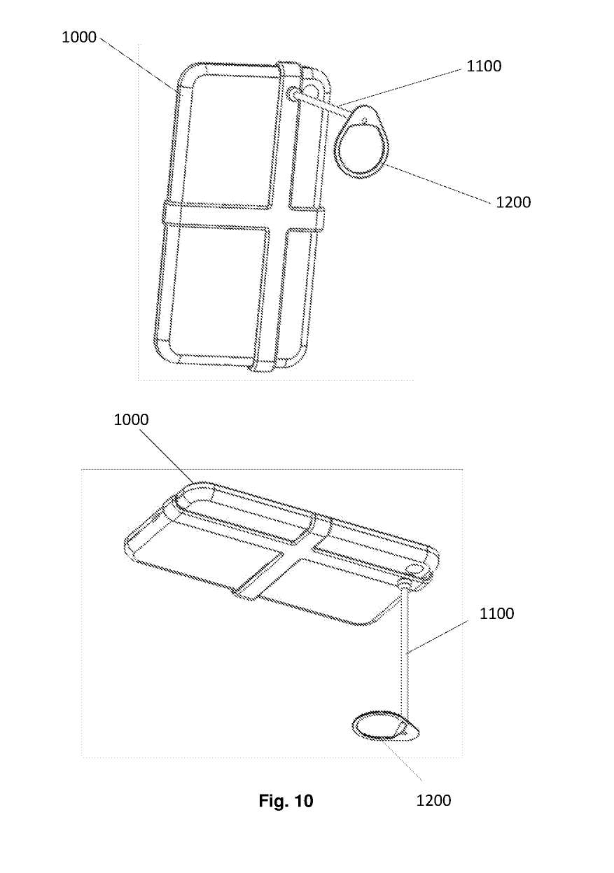

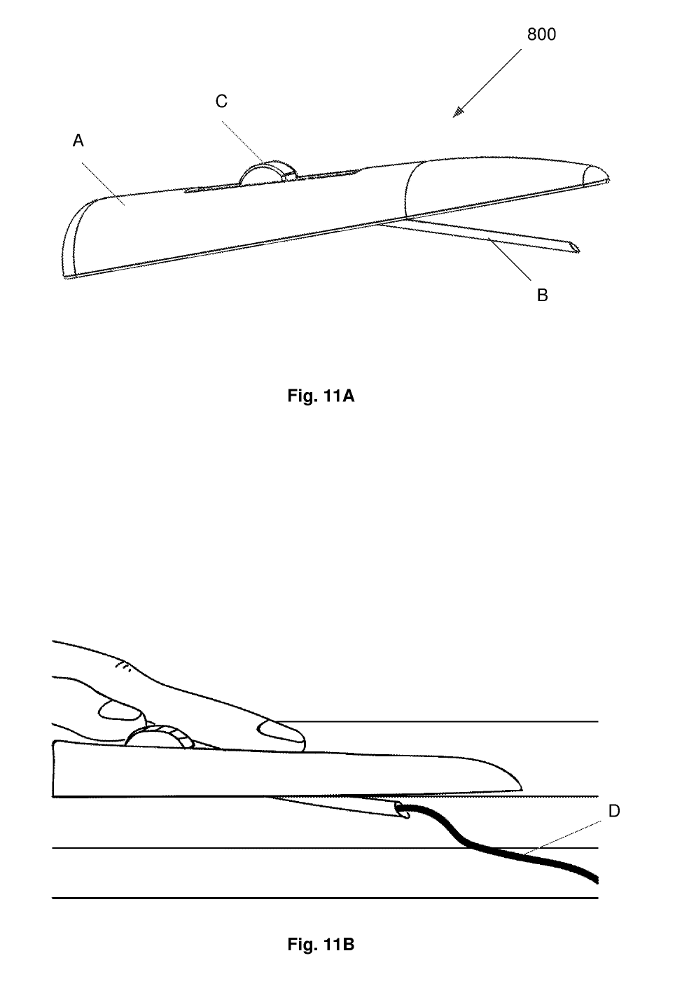

According to some embodiments, as seen in FIG. 7A, the light positioning device of the present invention 710 may be installed as an add-on to existing conventional TIVADs 720, already known to the medical staff, using existing surgical procedures for insertion. The added benefit is in providing illumination for easier targeting.

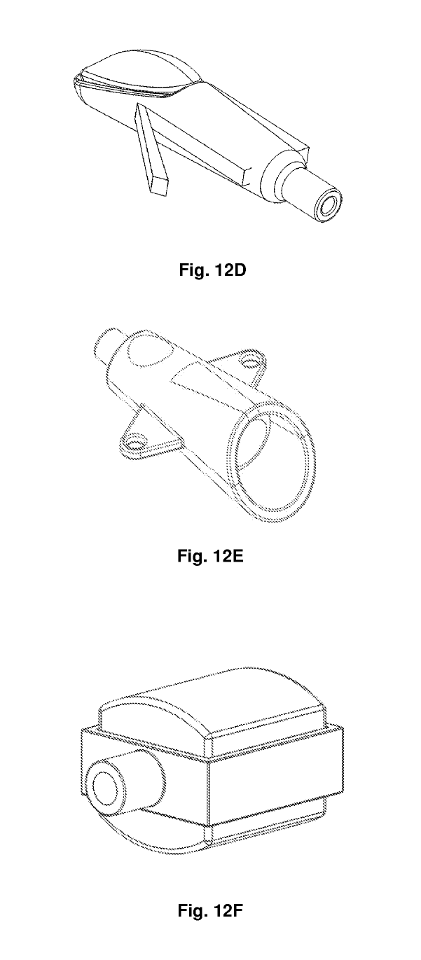

The target shape may be illuminated in various shapes, as shown in FIG. 7B. Light carrying channels (4 in FIGS. 1 and 10 in FIG. 6) may be implemented by covering pre-formed channels in the devices with a glass or plastic light reflecting layer. Preferably, the light carrying channels are implemented by optic fibers embedded in the devices.

TIVAD device 28 may be manufactured for example from Plastic or Titanium, preferably from a Plastic material. The diameter of housing 7 may generally be in the range of 0.5 to 1.5 cm, preferably about 0.75 cm, its length may generally be in the range of 0.5 to 2 cm, preferably about 1.5 cm, and its wall thickness may generally be in the range of 1 to 4 mm, preferably about 3 mm. Septum 8 may be manufactured from a piece of Silicone having a diameter of about 0.75 to 1 cm, and it can be sealably (securely) attached in, or over, whilst sealing an opening provided in housing 7 by means of conventional techniques, as well known in the TIVAD industry.

The TIVAD device according to the present invention may be constructed of fully transparent materials or of engraved optic boards, providing light reflection on all sides thereof.

It is noted that the sub-dermal location system of the invention, as explained and discussed herein above, can be implemented in other medical devices, not only with TIVADs, such as, but not limited to, dialysis grafts, ventriculo-peritoneal shunts and tissue expanders.

The TIVAD device of the invention enables a safe and easy access to the membrane (septum) of the device for chemotherapeutic agent delivery. If the TIVAD tilts or rotates after placement, it could be placed properly by viewing the current location and orientation of the TIVAD using the illumination means. It can especially assist patients with special skin such as obese patients. Furthermore, a colored filter integrated to the optical fibers can indicate the type of the port, such as power or non-power port.

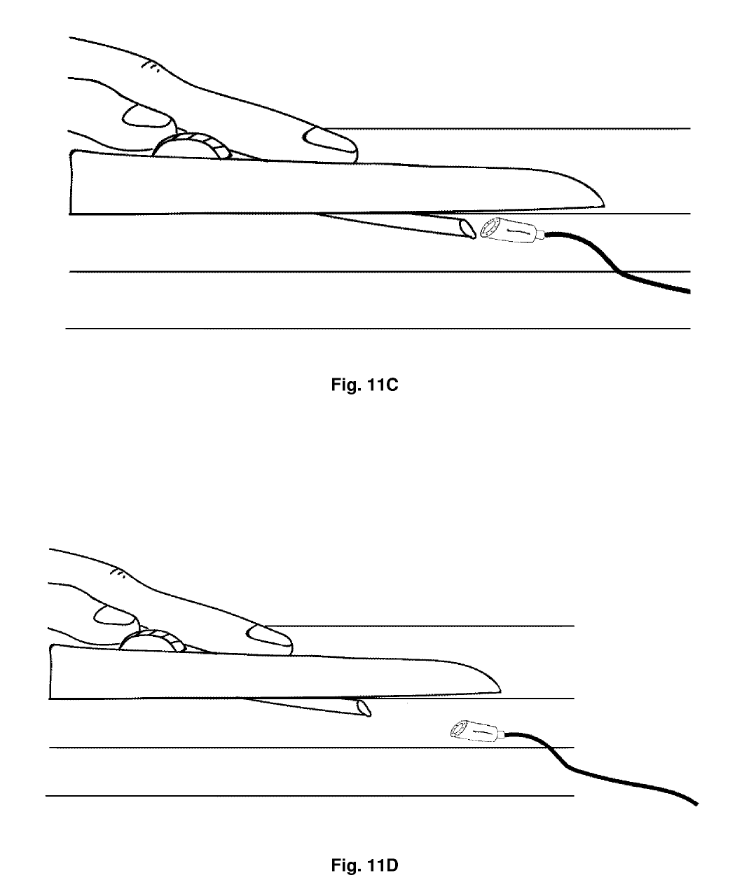

In another embodiment of the present invention, there is a designated access device comprising a needle (B) and integral light source (A), allowing lighting the skin to find the implanted port (FIG. 8) for injecting substances into the implanted TIVAD. The needle is of generic design and can be used with other central vascular access devices.

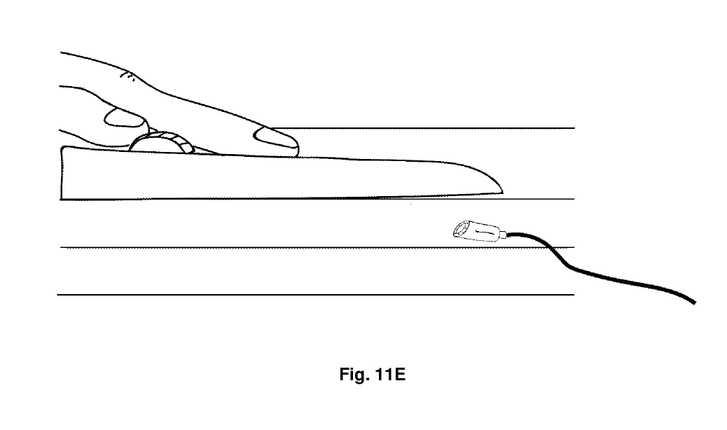

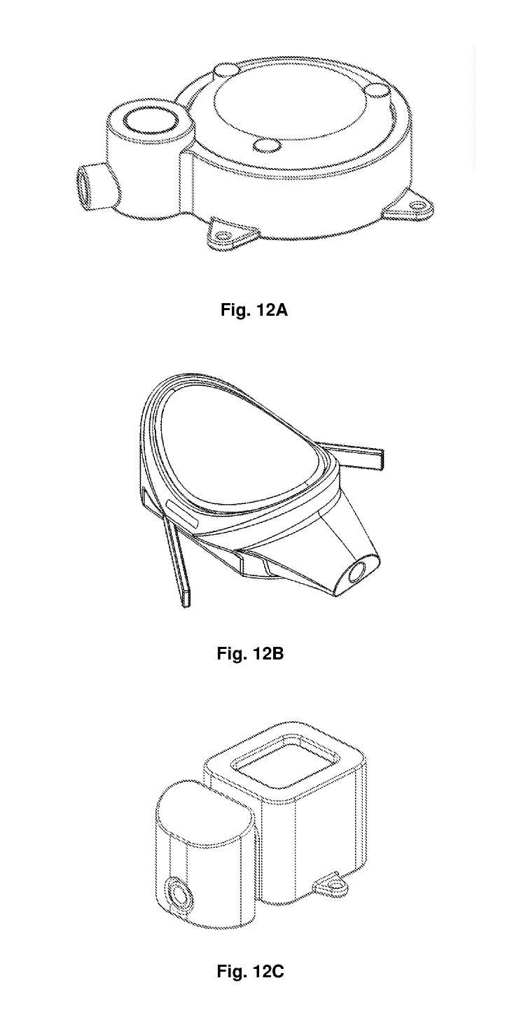

In another embodiment of the present invention, there is a designated light emitting device (FIGS. 9A-9B). The device provides another option for the lighting mechanism including a designated sterile patch (A) with a light source (B). The patch is held by the caregiver, near the skin of the patient, in search of reflected light. When light is noticed the patch is applied to the skin in a manner overlapping the patch outer orifices (C), and the inner orifice (D) marks the septum of the port location. The needle is then inserted through the inner orifice to the port.

In the embodiment of FIG. 9C the light emitting device is attached to the physician's hand, enabling convenient simultaneous illumination and injection.

All of the above mentioned features are given by way of example only, and may be changed in accordance with the differing requirements of the various embodiments of the present invention. Thus, the above mentioned features should not be construed as limiting the scope of the present invention in any way.

According to embodiments of the invention, a smartphone running a dedicated application may be used in conjunction with the TIVAD device for:
Collecting information when the TIVAD is in use, such as type of drug injected, date and time, etc.
Illuminating the skin and detecting the TIVAD.
Capturing the illuminated septum area.
Navigating the injecting device to areas less dense with previous injections, thus prolonging the usability of the septum.

This may be done, for example, as depicted in FIG. 10, by attaching a light guide 1100 to the LED phone flash. The other end of the optic fiber may optionally be connected to a ring 1200. In operation, the far end of the optic fiber (and the ring) is placed on the patient's skin, to cause illumination of the TIVAD. The ring then serves as guide for injecting the required substance into the TIVAD. In addition, it is to be appreciated that the different housings, light carrying channels, septums, and other members, described herein above may be constructed in different shapes (e.g. having oval, square etc. form in planar view) and sizes differing from those exemplified in the preceding description.

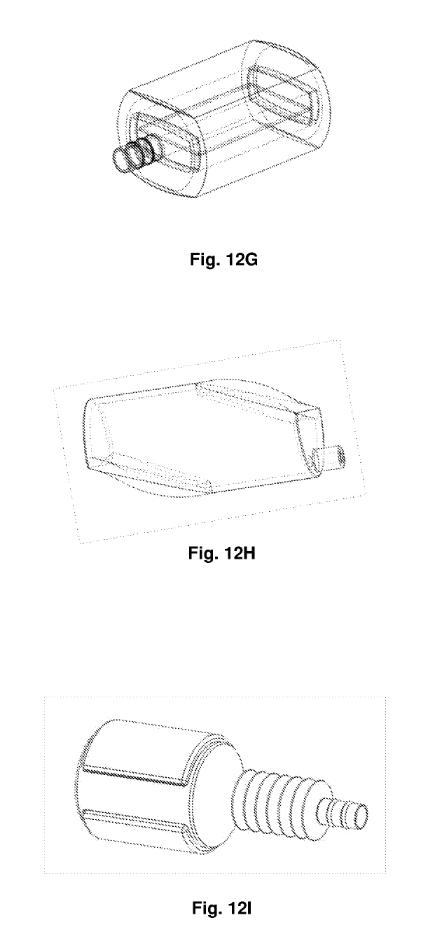

In another embodiment of the present invention, there is a designated implantation device 800 for precise subcutaneous implantation of the port (FIGS. 11A-11E). The implantation device works in a manner similar to injection. The device is comprised of a body (A), a needle (B) and an operation button (C) (FIG. 8A).

Before the implantation of the port, the catheter (D) is manually inserted using common procedures (FIG. 11B). The catheter is connected on its proximal side to the port, and they are loaded to the implantation device 800. The implantation device's needle punctures the skin, precise implantation depth is kept due to the distance relationship between the body of the implantation device and the needle (FIG. 11B). Following insertion, the needle is withdrawn, leaving the port secured in the subcutaneous tissue (FIGS. 11C-11E).

In an alternative embodiment, the catheter (D) may also be inserted using the implantation device.

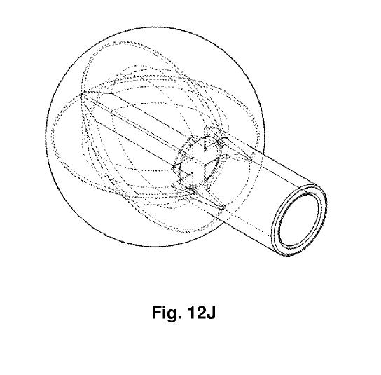

FIGS. 12A through 12J depict various exemplary designs of the TIVAD according to the present invention, partly or entirely transparent. The devices shown in FIGS. 12D and 12E have sleek shapes that can be injected in a non surgical operation. The devices shown in FIGS. 12F through 12I have two symmetric transparent septums with large injection surface on both sides, thus may be used even if flipped. The device shown in FIG. 12J has a spherical septum with a rigid core that prevents passage of the needle from one side to the other. This device may be used in any orientation.

While some of the embodiments of the invention have been described by way of illustration, it will be apparent that the invention can be carried into practice with many modifications, variations and adaptations, and with the use of numerous equivalents or alternative solutions that are within the scope of a person skilled in the art, without departing from the spirit of the invention.

1. A totally implantable medical device configured to be implanted under a patient's dermal layer for vascular access comprising:
a housing comprising a reservoir;
said reservoir connected on one end thereof to a self-resealing penetrable septum and on the other end to an outlet cannula; and
one or more inter-digitating light carrying channels connected with said housing having their openings circumferentially scattered about the outer rim of said septum, said one or more inter-digitating light carrying channels configured to internally transmit light rays by means of internal reflection.

2. (canceled)

3. The medical device of claim 1 wherein said one or more inter-digitating light carrying channels comprise optic fibers.

4. The medical device of claim 1 wherein said one or more inter-digitating light carrying channels comprise light guides.

5. The medical device of claim 1 wherein said one or more inter-digitating light carrying channels comprise light reflecting materials.

6. The medical device of claim 5 wherein said light reflecting materials are selected from the group consisting of: acrylic glass (PMMA), mirrors and other light reflecting materials.

7. The medical device of claim 1, wherein said septum is located on the side or upper surface of said housing.

8. The medical device of claim 1 further comprising integrated stabilization wings.

9. The medical device of claim 1 configured to be located using a generic light source.

10. The medical device of claim 1, comprising one of: tissue expander, vascular access device, vascular graft and dialysis/surgical bypass graft.

11. (canceled)

12. A designated access device configured to locate an implanted medical device according to claim 1, comprising:
a body; a needle connected to said body; and integral light source connected to said body.

13. A designated light emitting device configured to mark the septum of an implanted medical device according to claim 1, comprising: a sterile patch; said patch comprises outer orifices and an inner orifice; and a light source located on said patch.

14. A sub-dermal bundle of optical fibers configured to mark a desired location on the skin under induction of an external light source.

15. A method of locating a medical device, comprising one or more inter-digitating light carrying channels configured to internally transmit light rays by means of internal reflection, under a patient's dermal layer for vascular access, comprising:
inducing light with a light source on said patient's skin; and
finding a reflected light area from said one or more inter-digitating light carrying channels on said patient's skin.

16. (canceled)

17. A method of locating a medical device comprising inter-digitating light carrying channels, configured to internally transmit light rays by means of internal reflection, under a patient's dermal layer, comprising:
holding a sterile patch comprising a light emitting device near a patient's skin;
searching for reflected light; and
applying said patch to said patient's skin to overlap said reflected light when said reflected light is noticed.

18. A method of locating a desired location on the skin, comprising:
inserting sub-dermally a bundle of optical fibers;
inducing an external light source; and
searching for reflected light.

19. A method of locating a totally implantable vascular access device (TIVAD) comprising one or more inter-digitating light carrying channels under a patient's dermal layer, said one or more inter-digitating light carrying channels configured to internally transmit light rays by means of internal reflection, comprising:
launching a dedicated application running on a smartphone and operable to:
collect information when the TIVAD is in use;
illuminate the skin using the smartphone's LED;
detect reflected light from said one or more inter-digitating light carrying channels;
capture an image of said reflected light;
analyze the captured image; and
navigate an injecting device to areas less dense with previous injections.

20. The method of claim 19, wherein said information comprises at least one of type of drug injected, date and time.

21. The method of claim 20, wherein said illuminating comprises attaching a light guide to the LED phone flash.
